(19) United States
(12) Patent Application Publication
(10) Pub. No.: US 2021/0404887 A1
(43) Pub. Date: Dec. 30, 2021

(54) INTELLIGENT TEMPERATURE WARNING DEVICE APPLIED TO WEARABLE PRODUCTS

(71) Applicant: TERRY ELECTRONICS (S.Z) CO., LTD., Shenzhen (CN)

(72) Inventor: TINGDONG GOU, Shenzhen (CN)

(21) Appl. No.: 16/931,473

(22) Filed: Jul. 17, 2020

(30) Foreign Application Priority Data
Jun. 24, 2020 (CN) 202010595431.2

(51) Int. Cl. G01K 13/00; A61B 5/01; A61B 5/00

(52) U.S. Cl. CPC G01K 13/002; A61B 5/746; A61B 5/01

(57) ABSTRACT

An intelligent temperature warning device applied to wearable products is provided, which is used in the technology field of temperature sensing. By applying the intelligent temperature warning device to various wearable products, the temperature of human body can be monitored to achieve the effect of generating interesting interactions and the process of warning when human body is at high temperature.

### CROSS REFERENCE TO RELATED APPLICATIONS

This application claims priority to Chinese Patent Application No. 202010595431.2, filed Jun. 24, 2020, which is hereby incorporated by reference herein as if set forth in its entirety.

### BACKGROUND

### 1. Technical Field

The present disclosure generally relates to technical field of temperature sensing, and particularly to an intelligent temperature warning device applied to wearable products.

### 2. Description of Related Art

Temperature transducer is a kind of sensor that can sense the temperature and convert it into usable output signal. The existing temperature transducer has not put forward the technology of interaction with human body, which lacks the effect of real-time temperature monitoring of human body and interesting interaction, and has no good warning function when human body is at high temperature.

### BRIEF DESCRIPTION OF THE DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 a schematic structural block diagram of an embodiment of the intelligent temperature warning device applied to wearable products of the present invention.

FIG. 2 is a circuit schematic diagram of an embodiment of the control circuit of the intelligent temperature warning device applied to wearable products of the present invention.

FIG. 3 is a schematic flowchart of an embodiment of executing a warning method by the control circuit of the intelligent temperature warning device applied to wearable products of the present invention.

FIG. 4 is a schematic view of an assembly structure when an embodiment of the intelligent temperature warning device of the present invention is applied to a cap.

FIG. 5 is a schematic view of an assembly structure when an embodiment of the intelligent temperature warning device of the present invention is applied to a shoe.

FIG. 6 is a schematic view of an assembly structure when an embodiment of the intelligent temperature warning device of the present invention is applied to a garment.

### DETAILED DESCRIPTION

The disclosure is illustrated by way of example and not by way of limitation in the figures of the accompanying drawings, in which like reference numerals indicate similar elements. It should be noted that references to “an” or “one” embodiment in this disclosure are not necessarily to the same embodiment, and such references can mean “at least one” embodiment.

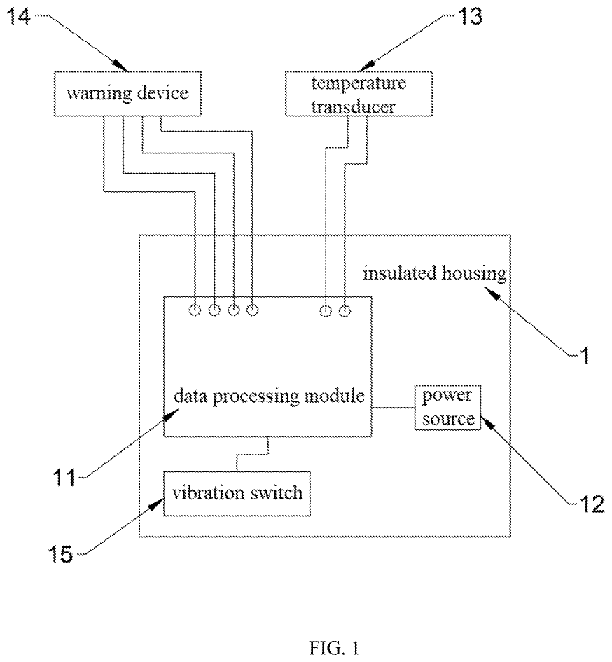

Referring to FIG. 1, a schematic structure block diagram of an intelligent temperature warning device applied to wearable products in an embodiment of the present invention is shown.

The intelligent temperature warning device includes a control circuit, a temperature transducer 13, a warning device 14 and a insulated housing 1.

The control circuit includes a power source 12, a data processing module 11 electrically connected to the power source 12, and a vibration switch 15 electrically connected to the data processing module 11. The vibration switch 15 vibrates to trigger the control circuit to conduct with the power source 12.

The temperature transducer 13 is electrically connected to the control circuit. The temperature transducer 13 collects temperature data and transmits it to the data processing module 11 of the control circuit, by which the data processing module 11 is processed and output.

The warning device 14 is electrically connected to the control circuit for outputting the warning information processed by the control circuit with the data processing module 11.

The insulated housing 1 is provided with an accommodating cavity, and the control circuit is insulated packaged in the accommodating cavity.

The temperature transducer 13 described above is a temperature sensing line.

In one embodiment, the warning device 14 is an LED illuminant, which can output different warning light effects respectively.

In one embodiment, the warning device 14 is a broadcasting unit, which can output different warning audios respectively.

Specifically, the data processing module 11 is connected to the power source 12 and has a first pin group and a second pin group.

The vibration switch 15 is connected to the data processing module 11 and starts temperature sensing of the temperature transducer under the premise of monitoring the vibration of the temperature control device.

The temperature transducer 13 is connected to the first pin group, and the data processing module 11 senses the temperature through the temperature transducer 13.

The warning device 14 is connected to the second pin group, receiving the control instruction output by the data processing module 11 and performing work.

Specifically, the data processing module 11 uses a single-chip control component such as MCU, which has a first pin group and a second pin group, and is connected to the power source 12. The power source 12 and the data processing module 11 are provided in the insulated housing 1. The temperature transducer 13 and the warning device 14 are both inserted into the insulated housing 1 and connected to the data processing module 11. The size of the insulated housing 1 is designed as a rectangular block, with a length of 1 cm˜2 cm, a width of 1 cm˜1.5 cm, and a height of 0.25 cm˜0.75 cm. It is easy to apply the intelligent temperature warning device for wearable products to a variety of wearable products through a smaller design. The temperature transducer has a sensing line to connect to the part of the wearable products that is close to human body to sense the temperature.

The above-mentioned first pin group is presented as a first pin and a second pin on the data processing module 11. One end of the temperature transducer 13 is connected to the first pin and the second pin, and the other end is extended outside the insulated housing 1 through wire to collect the temperature.

The above-mentioned second pin group is presented as a third pin, a fourth pin, a fifth pin and a sixth pin on the data processing module 11. One end of the warning device 14 is connected to the third pin, the fourth pin the fifth pin, and the sixth pin respectively, and the other end is extended outside the insulated housing 1 through wire to perform work.

In one embodiment, the warning device 14 includes an LED illuminant, and the specific process performed by the LED illuminant is as following.

The data processing module 11 obtains the sensed external temperature value through the temperature transducer 13.

The data processing module 11 identifies the temperature range in which the temperature value is located, and the temperature range includes a normal temperature range, a temperature increasing range, and a high temperature range. Specifically, the temperature range of the normal temperature range is 0° C. to 27° C. The temperature range of the temperature increasing range is 27.5° C. to 30° C. The temperature range of the high temperature range is above 30° C.

After determining the corresponding temperature range according to the temperature value, the data processing module 11 generates a control instruction corresponding to the temperature range and sends it to the LED illuminant, instructing the LED illuminant to output the corresponding illumination color. Specifically, the control instruction corresponding to the normal temperature range is to instruct the LED illuminant to output two cycles with green light, and the output cycle adopts a breathing flashing method, in which exhaling for 1 second and inhaling for 1 second to complete a cycle. The corresponding control instruction to the temperature increasing range is to instruct the LED illuminant to output two cycles with pink light, and the output cycle adopts a breathing flashing method, in which exhaling for 0.8 second and inhaling for 0.8 second to complete a cycle. The corresponding control instruction to the high temperature range is to instruct the LED illuminant to output two cycles with red light, and the output cycle adopts a breathing flashing method, in which exhaling for 0.5 second and inhaling for 0.5 second to complete a cycle.

In one embodiment, the warning device 14 includes a broadcasting unit, and the specific implementation process of the broadcasting unit is the same as that of the above-mentioned LED illuminant, specifically,

The data processing module 11 identifies the temperature range in which the temperature value is located, and the temperature range includes a normal temperature range, a temperature increasing range, and a high temperature range. Specifically, the temperature range of the normal temperature range is 0° C. to 27° C. The temperature range of the temperature increasing range is 27.5° C. to 30° C. The temperature range of the high temperature range is above 30° C.

After determining the corresponding temperature range according to the temperature value, the data processing module 11 generates a control instruction corresponding to the temperature range and sends it to the broadcasting unit, instructing the broadcasting unit to output the corresponding timbre or audio. Specifically, the timbre is preferably “tick”, and the audio frequency is the output frequency. The corresponding control instruction to the normal temperature range is to instruct the broadcasting unit not to output the timbre. The corresponding control instruction to the temperature increasing rang is to instruct the broadcasting unit to output the timbre, in which the output of the timbre adopts a “tick” sound interval of 1 second. The corresponding control instruction to the high temperature range is to instruct the broadcasting unit to output a timbre, in which the output of the timbre adopts a “tick” sound interval of 0.5 second.

The vibration switch 15 is energized when the temperature control device is vibrating, so that the power source 12 supplies power to the data processing module 11, and the data processing module 11 further performs temperature sensing through the temperature transducer 13.

In summary, the intelligent temperature warning devices can be assembled on the wearable products to monitor the user's body temperature in real time. whether the temperature control device vibrates can be determined through the vibration switch. If so, the control circuit obtains the user's body temperature value through the temperature transducer and generates a corresponding control instruction. The warning device performs the corresponding work according to the control instruction, so as to achieve the effect of generating a corresponding interesting interaction according to the user's body temperature.

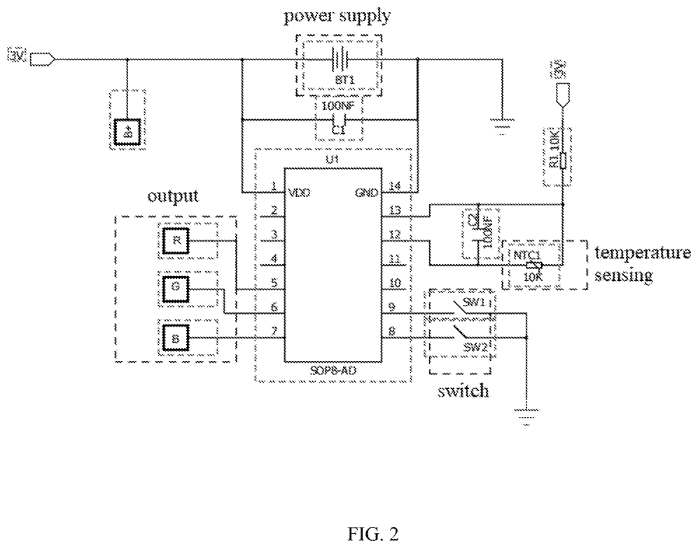

Referring to FIG. 2, a circuit schematic diagram of the intelligent temperature warning device provided by the present invention is shown. The control circuit includes a control module, a temperature sensing module, a vibration switch module, an output module and a power supply module. The temperature sensing module, the vibration switch module, the output module and the power supply module are connected to the control module respectively.

The control module has a 1^{st} leading point and a 14^{th} leading point. The power supply module comprises a battery pack and a first capacitor. The battery pack is connected in parallel with the first capacitor and connected to the 1^{st} leading point and the 14th leading point respectively.

The control module has a 5^{th} leading point, a 6^{th} leading point and a 7^{th} leading point, and the output module is connected to the 5^{th} leading point, the 6^{th} leading point and the 7^{th} leading point respectively.

The control module has an 8^{th} leading point and a 9^{th} leading point. The vibration switch module includes a first switch, a second switch, and a vibration sensing unit. The first switch and the second switch are connected in parallel to the 8^{th} leading point and the 9^{th} leading point. And both the first switch and the second switch are connected in series with the vibration sensing unit.

The control module has a 12^{th} leading point and a 13^{th} leading point. The temperature sensing module includes a temperature sensing unit, a second unit, and a resistor. The temperature sensing unit, the second unit, and the resistor are connected in series to the 12^{th} leading point and the 13^{th} leading point.

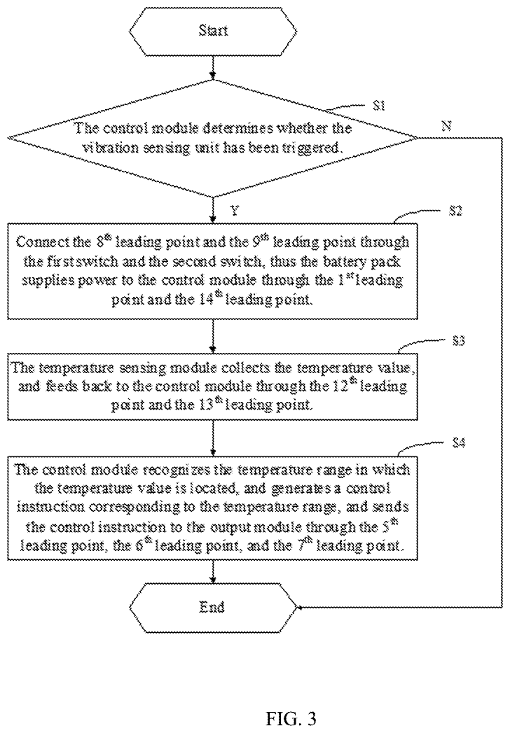

Referring to FIG. 3, a schematic flowchart of executing a warning method by a control circuit of the intelligent temperature warning device according to the present invention in shown. The warning method executed by the control circuit of the above-mentioned intelligent temperature warning device applied to wearable products includes the following steps.

S1, the control module determines whether the vibration sensing unit has been triggered;

S2, if so, connect the 8^{th} leading point and the 9^{th} leading point through the first switch and the second switch, thus the battery pack supplies power to the control module through the 1^{st} leading point and the 14^{th} leading point;

S3, the temperature sensing module collects the temperature value, and feeds back to the control module through the 12^{th} leading point and the 13^{th} leading point;

S4, the control module recognizes the temperature range in which the temperature value is located, and generates a control instruction corresponding to the temperature range, and sends the control instruction to the output module through the 5^{th} leading point, the 6^{th} leading point, and the 7^{th} leading point.

It should be noted that the above-mentioned vibration sensing unit includes a spring and a probe. The probe penetrates into the spring, and the spring and the probe are electrically connected. When the intelligent temperature warning device is stationary, the probe and the spring do not touch. When the intelligent temperature warning device is vibrated, the spring touches the probe and conducts to generate a signal. The signal is received by the control module, which determines that the vibration sensing unit is triggered, and performs the next step.

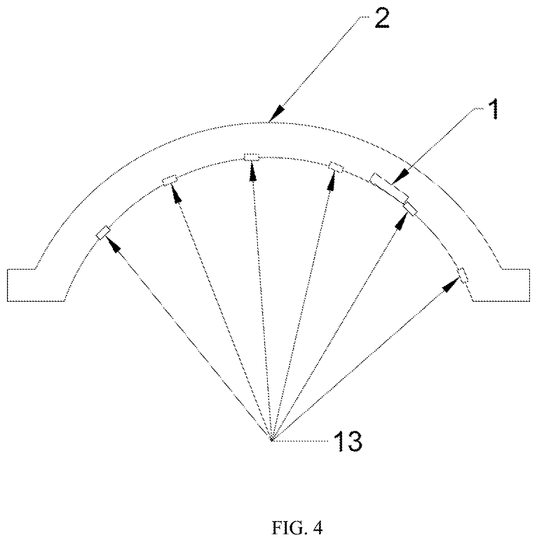

Referring to FIG. 4, a schematic structural view of a wearable product adopting a cap 2 according to the present invention is shown. The above-mentioned intelligent temperature warning device is applied to a cap 2, and the insulated housing 1 and the warning device 14 are provided on the cap.

The cap includes a concave portion, and the sensing end of the temperature transducer is disposed on the side wall of the concave portion to sense the temperature of human head. The sensing end of the temperature transducer 13 of the intelligent temperature warning device is disposed on the inner side of the cap 2 to sense the temperature of human head.

It should be noted that the present invention is not limited to a specific position where the warning device 14 of the intelligent temperature warning device is installed on the cap 2, such as a brim, a top of the cap, etc. The temperature transducer 13 is required to be disposed inside of the cap 2, to sense the temperature of human head, and then the data processing module 11 obtains the temperature value of human head to generate a corresponding instruction to instruct the warning device 14 to perform the corresponding operation, which improved interaction with human head.

In another embodiment, there are a plurality of temperature transducers 13 described above, which are distributed in various parts of the cap 2 facing inward to sense the user's head in all directions.

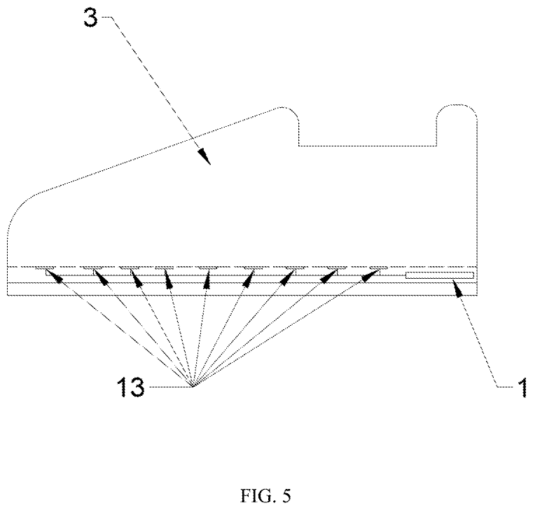

Referring to FIG. 5, a schematic structural view of a wearable product adopting a shoe 3 according to the present invention is shown. The above-mentioned intelligent temperature warning device is applied to the shoe 3, and the insulated housing 1 and the warning device 14 are provided on the shoe 3.

The shoe includes an insole portion, which is located at a position where the shoe is stepped on by human body. The sensing end of the temperature transducer is located on the insole portion to sense the temperature of human foot. The sensing end of the temperature transducer 13 is located on the insole of the shoe 3 where the shoe is stepped on to sense the temperature of human foot.

The present invention is not limited to a specific position where the warning device 14 of the intelligent temperature warning is installed on the shoe 3, such as the bottom, side, and edge of the shoe, etc. The temperature transducer 13 is required to be located at a position where the insole is stepped on to sense the temperature of human foot, and then the data processing module 11 obtains the temperature value of human foot to generate a corresponding instruction to instruct the warning device 14 to perform the corresponding operation, which improved interaction with human foot.

In another embodiment, there are a plurality of above-mentioned temperature transducers 13 distributed on various parts of the stepped portion of the insole of the shoe 3 to sense the user's foot in all directions.

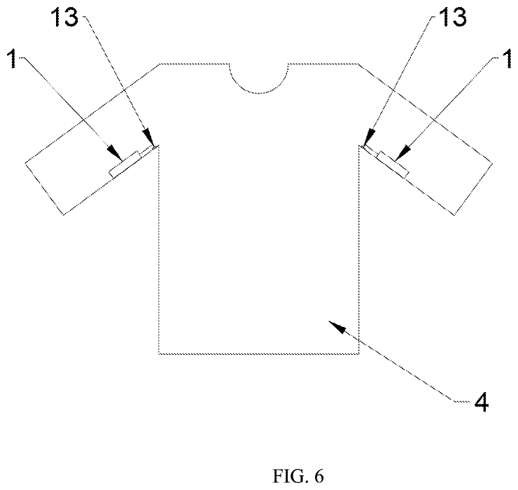

Referring to FIG. 6, a schematic structural view of a wearable product adopting a garment 4 according to the present invention is shown. The above-mentioned intelligent temperature warning device is applied to the garment 4, and the insulated housing 1 and the warning device 14 are provided on the garment 4.

The sensing end of the temperature transducer 13 is provided at the connection position between the sleeve and the garment body to sense the temperature of human armpit.

The present invention is not limited to a specific position where the warning device 14 of the intelligent temperature warning device is installed on the garment 4, such as the outer side and the edge of the garment 4, etc. The temperature transducer 13 is required to be provided at the connection between the sleeve and the garment body to sense the temperature of human armpit, and then the data processing module 11 obtains the temperature value of human armpit to generate a corresponding instruction to instruct the warning device 14 to perform the corresponding operation, which improved interaction with human body.

In another embodiment, there are two above-mentioned temperature transducers 13 respectively disposed at the two connection positions between the sleeves and the garment body to collect the temperature of the armpits of the user.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. An intelligent temperature warning device applied to wearable products, the intelligent temperature warning device comprising:
a control circuit comprising a power source, a data processing module electrically connected to the power source, and a vibration switch electrically connected to the data processing module, wherein the vibration switch vibrates to trigger the control circuit to conduct with the power source;
a temperature transducer electrically connected to the control circuit, collecting and transmitting temperature data to the data processing module of the control circuit, whereby the temperature data is processed and output; and
a warning device electrically connected to the control circuit for outputting the warning information processed by the control circuit with the data processing module; and
an insulated housing provided with an accommodating cavity, wherein the control circuit is insulated packaged in the accommodating cavity.

2. The intelligent temperature warning device of claim 1, wherein the temperature transducer is a temperature sensing line.

3. The intelligent temperature warning device of claim 1, wherein the warning device is an LED illuminant capable of outputting different warning light effects respectively.

4. The intelligent temperature warning device of claim 3, wherein the warning light effect output by the LED illuminant is selected from one of a color group including green, pink and red.

5. The intelligent temperature warning device of claim 4, wherein the LED illuminant outputs the warning light effect by a breathing flashing method, wherein,
when the warning light effect output by the LED illuminant is green, the LED illuminant exhales for 1 second and inhales for 1 second to complete a cycle, and cycle two times; wherein,
when the warning light effect output by the LED illuminant is pink, the LED illuminant exhales for 0.8 second and inhales for 0.8 second to complete one cycle, and cycle two times; and wherein,
when the warning light effect output by the LED illuminant is red, the LED illuminant exhales for 0.5 second and inhales for 0.5 second to complete one cycle, and cycle two times.

6. The intelligent temperature warning device of claim 4, wherein the data processing module generates a corresponding control instruction according to the temperature range which the temperature data located in, to instruct the LED illuminant to output the corresponding warning light effect, wherein the temperature range comprises:
a normal temperature range, wherein the data processing module instructs the LED illuminant to output green light when the temperature data is between 0° C. and 27° C. in the normal temperature range;
a temperature increasing range, wherein the data processing module instructs the LED illuminant to output pink light when the temperature data is between 27.5° C. and 30° C. in the temperature increasing range; and
a high temperature range, wherein the data processing module instructs the LED illuminant to output red light when the temperature data is above 30° C. in the high temperature range.

7. The intelligent temperature warning device of claim 1, wherein the warning device is a broadcasting unit capable of outputting different warning audios respectively.

8. The intelligent temperature warning device of claim 7, wherein the data processing module generates a corresponding control instruction according to the temperature range which the temperature data located in, to instruct the broadcasting unit to output the corresponding warning audio, wherein the temperature range comprises:
a normal temperature range, wherein the data processing module instructs the broadcasting unit to suspend the output of audio when the temperature data is between 0° C. and 27° C. in the normal temperature range; and
a temperature increasing range, wherein the data processing module instructs the sound broadcasting unit to output audio at intervals of 1 second when the temperature data is between 27.5° C. and 30° C. in the temperature increasing range;
a high temperature range, wherein the data processing module instructs the sound broadcasting unit to output audio at intervals of 0.5 second when the temperature data is above 30° C. in the high temperature range.

9. The intelligent temperature warning device of claim 1, wherein the control circuit comprises a control module, a temperature sensing module, a vibration switch module, an output module and a power supply module; and the temperature sensing module, the vibration switch module, the output module and the power supply module are connected to the control module respectively, wherein,
the control module has a 1^{st} leading point and a 14^{th} leading point, and the power supply module comprises a battery pack and a first capacitor, wherein the battery pack is connected in parallel with the first capacitor and connected to the 1^{st} leading point and the 14th leading point respectively; wherein,
the control module has a 5^{th} leading point, a 6^{th} leading point and a 7^{th} leading point, and the output module is connected to the 5^{th} leading point, the 6^{th} leading point and the 7^{th} leading point respectively; wherein,
the control module has an 8^{th} leading point and a 9^{th} leading point, and the vibration switch module comprises a first switch, a second switch, and a vibration sensing unit, wherein the first switch and the second switch are connected in parallel to the 8^{th} leading point and the 9^{th} leading point, and both the first switch and the second switch are connected in series with the vibration sensing unit; and wherein
the control module has a 12^{th} leading point and a 13^{th} leading point, and the temperature sensing module comprises a temperature sensing unit, a second unit, and a resistor, wherein the temperature sensing unit, the second unit, and the resistor are connected in series to the 12^{th} leading point and the 13^{th} leading point.

10. The intelligent temperature warning device claim 9, wherein the control circuit of the intelligent temperature warning device executes a warning method, and the method comprises:
the control module determining whether the vibration sensing unit has been triggered;
if yes, connecting the 8^{th} leading point and the 9^{th} leading point through the first switch and the second switch, thus the battery pack supplying power to the control module through the Pt leading point and the 14^{th} leading point;
the temperature sensing module collecting the temperature value, and feeding back to the control module through the 12^{th} leading point and the 13^{th} leading point; and
the control module recognizing the temperature range the temperature value located in, generating a control instruction corresponding to the temperature range, and sending the control instruction to the output module through the 5^{th} leading point, the 6^{th} leading point, and the 7^{th} leading point.

11. The intelligent temperature warning device of claim 1, wherein the intelligent temperature warning device is applied to a cap, and the insulated housing and the warning device are provided on the cap;
wherein the cap comprises a concave portion, and a sensing end of the temperature transducer is disposed on a side wall of the concave portion to sense the temperature of human head.

12. The intelligent temperature warning device of claim 1, wherein the intelligent temperature warning device is applied to a shoe, and the insulated housing and the warning device are provided on the shoe;
wherein the shoe comprises an insole portion located at a position where the shoe is stepped on by human body, and a sensing end of the temperature transducer is located on the insole portion to sense the temperature of human foot.

13. The intelligent temperature warning device of claim 1, wherein the intelligent temperature warning device is applied to a garment, and the insulated housing and the warning device are provided on the garment;
wherein a sensing end of the temperature transducer is provided at the connection position between the sleeve and the garment body to sense the temperature of human armpit.
